(19) United States
(12) Patent Application Publication
(10) Pub. No.: US 2020/0281989 A1
(43) Pub. Date: Sep. 10, 2020

(54) METHOD OF PREPARING A COMPOSITION FOR TREATING CANCER

(71) Applicants: JASSIM M. HASSAN M. ALI, SAFAT (KW); PEIYING YANG, SAFAT (KW); CECIL PACE-ASCIAK, SAFAT (KW)

(72) Inventors: JASSIM M. HASSAN M. ALI, SAFAT (KW); PEIYING YANG, SAFAT (KW); CECIL PACE-ASCIAK, SAFAT (KW)

(21) Appl. No.: 16/809,385

(22) Filed: Mar. 4, 2020

Related U.S. Application Data
(60) Provisional application No. 62/813,808, filed on Mar. 5, 2019.

(51) Int. Cl. A61K 35/60; A61K 9/16

(52) U.S. Cl. CPC A61K 35/60; A61K 9/1617

(57) ABSTRACT

A method of preparing a composition for treating cancer can include collecting epidermal gel secretions of catfish, extracting the total lipid fraction from the freeze-dried epidermal gel secretions to provide a total lipid fraction including neutral lipids, glycolipids, and phospholipids. A neutral lipid fraction, a glycolipid fraction, and a phospholipid fraction can then be separated from the total lipid fraction. Each of the total lipid fraction, the neutral lipid fraction, the glycolipid fraction, and the phospholipid fraction can exhibit anti-inflammatory and/or anti-cancer activities.

### CROSS-REFERENCE TO RELATED APPLICATION

This application claims the benefit of U.S. Provisional Patent Application Ser. No. 62/813,808, filed Mar. 5, 2019.

### BACKGROUND

### 1. Field

The disclosure of the present patent application relates to the use of preparations from the epidermal gel secretions (EGS) of catfish for therapeutic purposes, and particularly, for treating cancer.

### 2. Description of the Related Art

Cancer is a disease that is very difficult to cure when considering the cost, side effects, toxicity, and variable effectiveness of available treatment drugs.

The Arabian Gulf catfish (Arius bilineatus, (Valenciennes) naturally secretes a gel-like material (“epidermal gel secretion” or “EGS”) from its epidermis upon stress or injury. The epidermal gel secretion includes a complex mixture of biochemically and pharmacologically active lipids and proteins. Often times, however, the Arabian Gulf catfish (Arius bilineatus, (Valenciennes) produces venoms from its venomous spines and venom glands near its pectoral spines which mix with secretions on the catfish skin. Additionally, since the gelatinous secretion is exuded while the catfish is still alive, contaminants other than the venom (such as feces, vomit and blood) are also often mixed with the epidermal secretion.

Thus, a method of preparing a composition for treating cancer solving the aforementioned problems is desired.

### SUMMARY

A method of preparing compositions for treating cancer can include collecting epidermal gel secretions of catfish, freeze-drying the epidermal gel secretions, extracting the total lipid fraction (hereinafter, “CSP-L” or “total lipid fraction”) from the freeze-dried epidermal gel secretions to provide a total lipid fraction which includes the following three lipid fractions: neutral lipids, glycolipids, and phospholipids. The total lipid fraction can then be fractionated into a neutral lipid fraction, a glycolipid fraction, and a phospholipid fraction. Each of the lipid fractions (the neutral lipid fraction, the glycolipid lipid fraction, and the phospholipid lipid fraction) can exhibit anti-inflammatory and/or anti-cancer activities.

These and other features of the present disclosure will become readily apparent upon further review of the following specification and drawings.

### BRIEF DESCRIPTION OF THE DRAWINGS

FIG. 1A is a graph showing lipid fraction Fr6 inhibition of cell growth of human non-small cell lung cancer cells A549 (A).

FIG. 1B is a graph showing lipid fractions (CSP-L, Fr4 and Fr6) inhibition of cell growth of human pancreatic cancer cells (Panc-1 cells) and hepatocellular carcinoma cells (Hep3B).

FIG. 1C is a graph showing lipid fractions Fr4 (neutral lipids) and Fr6 (glycolipids) inhibition of cell growth of human prostate cancer cells (LNCaP cells).

FIG. 1D is a graph showing lipid fractions CSP-L (total lipids), Fr4 (neutral lipids), and Fr6 (glycolipids), inhibition of cell growth of human hepatocellular carcinoma cells Hep3B.

FIG. 2A is a graph showing anti-proliferative (cell growth inhibition) of human lung cancer NSCLC A549 cells by CSP-L (old total lipid fraction), Fr2 (new total lipid fraction), Fr3 (neutral lipid fraction produced by 75% chloroform chromatography column separation), Fr4 (neutral lipid fraction produced by 100% chloroform chromatography column separation), Fr5 (glycolipid lipid fraction produced by 75% acetone chromatography column separation) and Fr6 (glycolipid lipid fraction produced by 100% acetone chromatography column separation).

FIG. 2B is a graph showing anti-proliferative effects on liver cancer Hep3B cells by CSP-L (old total lipid fraction), Fr2 (new total lipid fraction), Fr3 (neutral lipid fraction produced by 75% chloroform chromatography column separation), Fr4 (neutral lipid fraction produced by 100% chloroform chromatography column separation), Fr5 (glycolipid lipid fraction produced by 75% acetone chromatography column separation) and Fr6 (glycolipid fraction produced by 100% acetone chromatography column separation.

FIG. 3A is a graph showing the effect of Fr3 (neutral lipids) on human melanoma MEWO cells.

FIG. 3B is a graph showing dose/response of fraction 3 inhibiting leukemia K562 cell survival in synergism with Gleevec. The graph shows the effect of Gleevec alone, Fr3 alone, and Gleevec in combination with fraction Fr3.

FIG. 4A is a graph showing the effect of Fr3 (neutral lipids) and the two isolated components from it, furan (F6), and a steroid (S5), on leukemic cells K562 using the WST-1 spectrophotometric detection assay.

FIG. 4B is a graph showing the effect of fraction 3, the two isolated components from it, furan (F6) and a steroid (S5), on breast MCF-7 using the WST-1 spectrophotometric detection assay.

FIG. 4C is a graph showing the effect of fraction 3, the two isolated components from fraction 3, furan (F6) and a steroid (S5), on human breast cancer cells MDA MB-231 using the WST-1 spectrophotometric detection assay.

FIG. 5A is a graph showing the effect of the vehicle control on the cell cycle of human breast cancer MDA-MB-231 cells.

FIG. 5B is a graph showing the effect of the furan fatty acid (F6) on the cell cycle of human breast cancer MDA-MB-231 cells (cells were treated with furan fatty acid (F6) for 24 hrs).

FIG. 5C is a graph comparing the effects of different concentrations of the vehicle control, furan fatty acid F6 (10 μg/mL), and F6 (50 μg/mL) on human cell cycle analysis on 24 hr F6-treated breast cancer MDA-MB-231 cells.

FIG. 6A is a Western blot of proapoptotic proteins, cleaved PARP and total and cleaved Caspase-3 in F6-treated MDA-MB-231 cells.

FIG. 6B is a graph showing total and cleaved Caspase-3 in F6-treated MDA-MB-231 cells.

FIG. 6C is a graph showing cleaved PARP in F6-treated MDA-MB-231 cells.

FIG. 7A is a Western blot showing the expression of Caspase-7 in F6 treated MDA-MB-231 cells.

FIG. 7B is a graph showing the expression of PARP, caspase-9 and β-actin in F6 treated MDA-MB-231 cells.

FIG. 7C is a graph showing caspase-7 protein expression in F6 treated MDA-MB-231 cells.

FIG. 7D is a graph showing PARP and Caspase-9 protein expression in F6-treated MDA-MB-231 cells.

FIG. 8A is a graph showing the effect of the vehicle control on the cell cycle of human leukemia K562 cells.

FIG. 8B is a graph showing the effect of the furan fatty acid (F6) on the cell cycle of human leukemia K562 cells (cells were treated with furan fatty acid (F6) for 24 hrs).

FIG. 8C is a graph showing cell cycle analysis of F-6 treated human leukemia K562 cells comparing the effects of the vehicle control to F6 (1 ug/uL), and F6 (5 ug/uL).

FIG. 9A is a Western blot analysis of proapoptotic proteins, cleaved PARP, and cleaved Caspase-3 in F6-treated K562 cells.

FIG. 9B is a graph showing protein expression of cleaved PARP and Caspase-3 in F6-treated K562 cells.

FIG. 10A is a graph showing cell cycle analysis of Panc-1 cells treated with F6.

FIG. 10B is a graph showing the average cell count for invasion of Panc-1 cells treated with F6 (* p<0.05 treated versus control).

FIG. 11A is a graph showing the effect of Fraction 1 and fractions derived therefrom (fractions 3-7) on the growth of human NSCLC A549 cells.

FIG. 11B is a graph showing the effect of Fraction 1 (CSP-L, total lipids) and fractions derived therefrom (fractions 3-7) on the growth of Hep3B cells.

FIG. 11C is a graph showing the percentage of cells in a population of Hep3B cells treated with Fraction 1 (CSP-L, total lipids) (CSP-L inhibited proliferation of Hep3B cells by arresting the cells at the G1 growth phase).

FIG. 11D is a Transmission Electron Microscope (TEM) image of vehicle control cells (5,000×).

FIG. 11E is a Transmission Electron Microscope (TEM) image of CSP-L-treated cells (5,000×).

FIG. 11F is a Transmission Electron Microscope (TEM) image of vehicle control cells (25,000×).

FIG. 11G is a Transmission Electron Microscope (TEM) image of CSP-L-treated cells (25,000×) (the black arrow indicates autophagosomes).

FIG. 12A is a graph showing the effects of Fraction 1 (CSP-L total lipids) on pancreatic cancer type Panc-1 cells and Hep3B cells in 21) culture.

FIG. 12B is a graph showing the effects of Fraction 1 (CSP-L total lipids) on Hep3B cells in 3D culture.

FIG. 12C is a graph showing the effects of Fraction 1 (CSP-L total lipids) on the cell cycle of pancreatic cancer type Panc-1 cells in 3D culture.

FIG. 12D is a graph showing the effects of Fraction 1 (CSP-L total lipids) on the cell cycle of Hep3B cells.

FIG. 12E is a graph showing the effects of Fraction 1 (CSP-L total lipids) on the cell cycle of pancreatic cancer type Panc-1 cells.

FIG. 13A is a graph showing the effects of Fraction 1 on the inhibition of metastatic potential of Hep3B cells.

FIGS. 13B-E are Transmission Electron Microscope (TEM) images of (FIG. 13B) control (0.1% ethanol) treated cells; (FIG. 13C) Fraction 1 (CSP-L, total lipids) in ethanol 5 μg/mL; (FIG. 13D) Fraction 1 (CSP-L, total lipids) in ethanol mixture 10 μg/mL; and (FIG. 13E) Fraction 1 (CSP-L, total lipids) in ethanol 25 μg/mL, respectively.

FIG. 13F is a graph showing the effects of Fraction 1 (CSP-L, total lipids) on the inhibition of metastatic potential of Panc-1 cells.

FIGS. 13G-I are Transmission Electron Microscope (TEM) images of (FIG. 13G) control (0.1% ethanol) treated cells; (FIG. 13H) Fraction 1 (CSP-L, total lipids) in ethanol 5 μg/mL; (FIG. 131) Fraction 1 (CSP-L, total lipids) in ethanol 10 μg/mL, respectively.

FIGS. 14A-14B are protein quantitation results showing that Fraction 1 (CSP-L, total lipids) modified cell cycle and cell signaling proteins in (FIG. 14A) Hep3B cells and (FIG. 14B) Panc-1 cells.

FIG. 15A is a graph showing that increasing concentrations of Fraction 1 (CSP-L, total lipids) inhibit protein abundance associated with the cell's normal activities such as cell cycle, metabolism, and cell signaling in Panc-1 cells (altered protein levels were measured by protein quantification array).

FIG. 15B is a Western blot for protein quantitation of acetyl CoA Carboxylase (ACC), eF2K, and SCD in Panc-1 cells (*p<0.05 treated versus control suggesting the statistical difference).

FIG. 15C is a graph showing the relative protein expression for the effect of Fraction 1 (CSP-L, total lipids) on acetyl CoA Carboxylyase (ACC), eEF2K, and SCD in Panc-1 cells.

FIGS. 16A-16B show (FIG. 16A) Western blot results showing that CSP-L alters cancer stem cell markers in Panc-1 cells and (FIG. 16B) a graph showing relative protein expression indicating that Fraction 1 (CSP-L, total lipids) alters cancer stem cell markers in Panc-1 cells (*p<0.05 treated versus control suggesting the statistical difference).

FIGS. 17A-17B are graphs showing the effect of two total lipid preparations, CSP-L-1 (old total lipid preparation, aged) and CSP-L-2 (new total lipid preparation), on (FIG. 17A) cell migration and (FIG. 17B) cell invasion (***p<0.01, ***p<0.005 treated versus control suggesting the statistical difference). Two major active components F6 and 3,5-cholestadiene found in CSP-L-1 and CSP-L-2, inhibit Hep3B cell metastasis (markers for A, migration and B, invasion).

FIGS. 17C-17E show Transmission Electron Microscope (TEM) images of the inhibitory effects of the control (FIG. 17C); CSP-L-1 (FIG. 17D); and CSP-L-2 (FIG. 17E).

FIG. 18A is a graph showing that F6 (found in Fraction 1) potently inhibited proliferation of human pancreatic cancer, Panc-1, Capan 2, and BxPC3 cells when these cells were treated with F6 (5-50 μg/ml) for 72 hours.

FIG. 18B is a graph showing the cell cycle analysis of control Panc-1 cells.

FIG. 18C is a graph showing the cell cycle analysis of Panc-1 cells treated with F6 (50 μg/ml).

FIG. 181) is a graph showing the cell cycle phase when Panc-1 cells were treated with F6 at different concentrations.

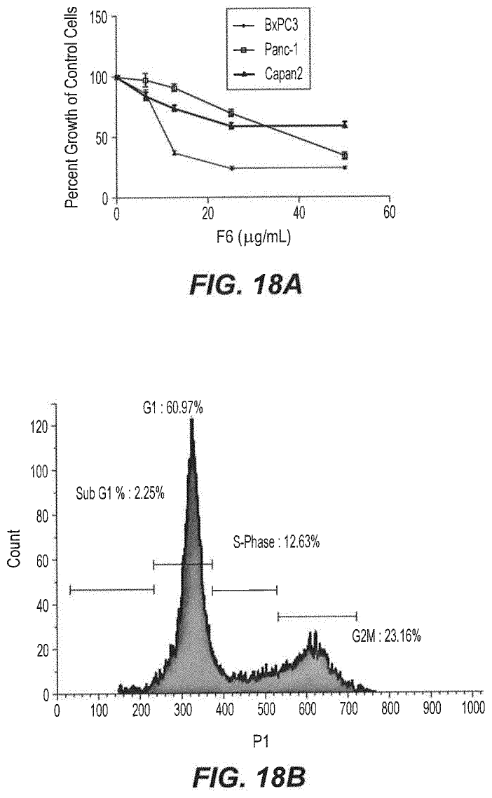

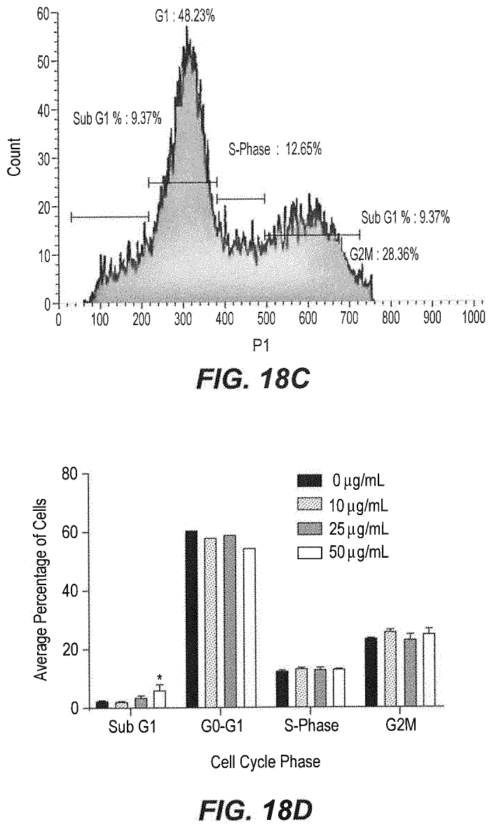

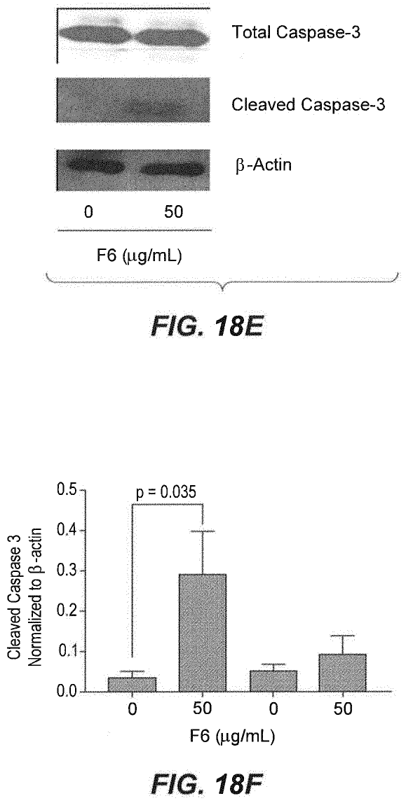

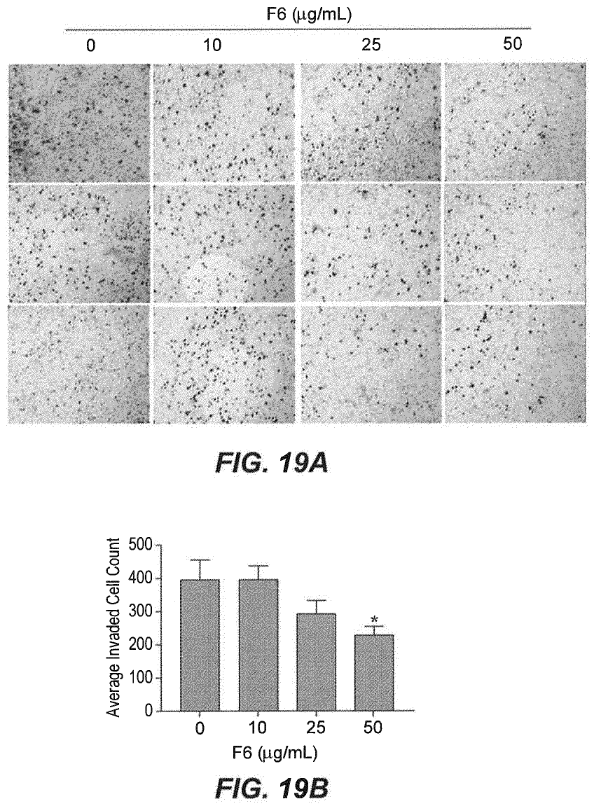

FIG. 18E depicts expression of proapoptotic proteins, cleaved caspase-3, and total caspase-3 in F6 treated Panc-1 cells at 24 hours (data are presented as means±SD * p<0.05 versus vehicle control treated).

FIG. 18F is a graph showing levels of caspase-3 normalized to β-actin when Panc-1 cells were treated with F6 at different concentrations.

FIG. 19A is a table of light microscopy images showing that F6 inhibited invasion of Panc-1 cells in a concentration dependent manner.

FIG. 19B is a graph showing the relationship between F6 concentration and average invaded cell count.

Similar reference characters denote corresponding features consistently throughout the attached drawings.

### DETAILED DESCRIPTION OF THE PREFERRED EMBODIMENTS

A method of preparing a composition for treating cancer can include collecting epidermal gel secretions of catfish, extracting the total lipid fraction (catfish skin preparation lipid fraction, CSP-L) from the freeze-dried epidermal gel secretions to provide a total lipid fraction including the neutral lipids, glycolipids, and phospholipids. A neutral lipid fraction, a glycolipid lipid fraction, and a phospholipid fraction can then be fractionated from the total lipid fraction. Each of the total lipid fraction, neutral lipid fraction, glycolipid fraction, and phospholipid fraction can exhibit anti-inflammatory and/or anti-cancer activities.

The first lipid fraction (also referred to as “CSP-L” or “total lipid fraction”) can be extracted from the freeze-dried epidermal gel secretions using a first extraction solvent. The first fraction or total lipid fraction can include neutral lipids, glycolipids, and phospholipids. The first extraction solvent can include chloroform, methanol and isopropanol. In an embodiment, the first extraction solvent includes chloroform, methanol and isopropanol in a ratio of 2:1:0.25 (v/v). Carbon dioxide (CO₂) as a supercritical fluid can also be used for total lipid extraction from freeze-dried CSP.

The extracted total lipids (CSP-L) can be fractionated into three classes of lipids, neutral lipids, glycolipids, and phospholipids. The fractionation of the three classes of lipids can be achieved by the use of column chromatography employing silica gel as a stationary phase packing material, packed in petroleum ether. CSP-L can be dissolved in a minimum volume of the extraction solvent system and loaded on the column, then eluted with three different solvent systems in sequence. Each solvent system can elute the lipid class it is intended to release. The first solvent system is for elution of the neutral lipids. The second solvent system is for elution of the glycolipids. The third solvent system is for elution of the phospholipids.

In an embodiment, the lipid fractionation can include packing a chromatography column with silica gel in petroleum ether to provide a stationary phase for separation of the three lipid classes. The column is then eluted with a series of solvent systems in sequence. It should be understood that each solvent system can elute only one class of lipids from the CSP-L and each class of lipids is eluted with a different solvent system. Each eluted lipid class fraction can be effective in cancer treatment, as described herein.

The neutral lipids are the least polar lipids and can be eluted first. Elution of the neutral lipids can include the use of chloroform. Since the column is previously packed with petroleum ether, a gradual change of elution solvent can be made from petroleum ether to chloroform. Otherwise the silica gel packing can be disrupted with cracking of the silica gel and, as a result, will fail to provide sufficient separation of lipid classes.

The second class of lipids that can be eluted after the neutral lipids are the more polar glycolipids. Elution of the glycolipids can include the use of acetone. Since the column has previously been filled with the chloroform for eluting the neutral lipids, a gradual change of elution solvent can be made from chloroform to acetone. Otherwise the silica gel packing can be disrupted with cracking of the silica gel and, as a result, will fail to provide sufficient separation of lipid classes.

The third class of lipids that can be eluted after the glycolipids are the phospholipids, the most polar of the lipids. Elution of the phospholipids can include the use of methanol. Since the column is full of acetone after separation of the glycolipids, a gradual change of elution solvent can be made from acetone to methanol. Otherwise, the silica gel packing can be disrupted with cracking of the silica gel and, as a result, will fail to provide sufficient separation of lipid classes.

After significant experimentation, the present inventors successfully determined the type of organic solvent and the ratio of solvent mixtures for achieving optimal separation of lipid classes.

In an embodiment, elution of the neutral lipid fraction can include dispensing the total lipid extract (Fraction 1; CSP-L) in a 30×2 cm Woelm Pharma silica gel column (70-100 mesh, 100-200 μm particle size), packed in petroleum ether. The column can be first eluted with 1 L petroleum ether. The elution can be continued with addition of an increasing amount of chloroform in petroleum ether in a linear gradient fashion as follows:

petroleum ether (1 liter);

petroleum ether: chloroform (75:25, v/v 250 ml);

petroleum ether: chloroform (50:50, v/v 500 ml); and

chloroform (100%) 1 liter.

The eluted solvents can be pooled and evaporated on a rotary evaporator at room temperature to produce Fraction 2, the neutral lipid fraction. The neutral lipid fraction can be stored under nitrogen in the dark at −80° C.

In an embodiment, elution of the neutral lipid fraction using the first solvent system can include a first eluting phase, wherein the first solvent system comprises 100% petroleum ether, a second eluting phase after the first eluting phase, wherein chloroform is added to the column in an amount sufficient to achieve a petroleum ether: chloroform ratio by volume of 75:25 in the first solvent system, a third eluting phase after the second eluting phase, wherein chloroform is added to the column in an amount sufficient to achieve a petroleum ether: chloroform ratio by volume of 50:50 in the first solvent system, and a fourth eluting phase after the third eluting phase, wherein chloroform is added to the column in an amount sufficient to achieve 100% chloroform in the first solvent system.

In an embodiment, elution of the glycolipid fraction can occur after elution of the neutral lipid fraction. Elution of the glycolipid fraction can include eluting the column with one liter chloroform followed by gradual addition of an increasing amount of acetone in chloroform in a linear gradient fashion as follows:

a) chloroform (1 liter) or chloroform:acetone (90:10, v/v)

b) chloroform: acetone (75:25, v/v) 250 ml

c) chloroform: acetone (50:50, v/v) 500 ml

d) acetone (100%) (1 liter)

In an embodiment, eluting the glycolipid fraction can include a first eluting phase, wherein the second solvent system comprises one liter of chloroform or a chloroform:acetone ratio by volume of 90:10, a second eluting phase after the first eluting phase, wherein acetone is added to the column in an amount sufficient to achieve a chloroform:acetone ratio by volume of 75:25 in the second solvent system, a third eluting phase after the second eluting phase, wherein acetone is added to the column in an amount sufficient to achieve a chloroform:acetone ratio by volume of 50:50 in the second solvent system, a fourth eluting phase after the third eluting phase, wherein acetone is added to the column in an amount sufficient to achieve 100% acetone in the second solvent system. The eluted organic solvents obtained from each eluting phase can be pooled and evaporated on a rotary evaporator at 25° C. to produce Fraction 3 lipids, the glycolipids. The glycolipids can then be stored under nitrogen in the dark at −80° C.

In an embodiment, elution of the phospholipid fraction can occur after elution of the glycolipid fraction. Elution of the phospholipid fraction can include eluting the column with acetone (e.g., 1 liter) followed by gradual addition of an increasing amount of methanol in acetone in a linear gradient fashion as follows:

a) acetone (1 liter)

b) acetone: methanol (75:25, v/v) 250 ml

c) acetone: methanol (50:50, v/v) 500 ml

d) methanol (100%) (1 liter)

In an embodiment, eluting the phospholipid fraction can include a first eluting phase, wherein the third solvent system comprises one liter of acetone, a second eluting phase after the first eluting phase, wherein methanol is added to the column in an amount sufficient to achieve a acetone:methanol ratio by volume of 75:25 in the third solvent system, a third eluting phase after the second eluting phase, wherein methanol is added to the column in an amount sufficient to achieve a acetone:methanol ratio by volume of 50:50 in the third solvent system, a fourth eluting phase after the third eluting phase, wherein methanol is added to the column in an amount sufficient to achieve 100% methanol in the third solvent system. The eluted organic solvents obtained from each eluting phase can be pooled and evaporated on a rotary evaporator at 25° C. to produce the phospholipid fraction. The phospholipids can then be stored under nitrogen in the dark at −80° C.

The lipid fractions can include at least some of the following components:

F6 fatty acid (or 12,15-epoxy-13,14-dimethyleicosa-12,14-dienoic acid)-a furanoid fatty acid found mostly in the neutral lipid fraction but also to a lesser extent in the other lipid fraction with anti-inflammatory and anti-proliferative activities;

S5 (or cholesta-3,5-diene)—a cholesterol metabolite found mostly in the neutral lipid fraction with anti-proliferative activities;

F3—a subfraction from the neutral lipids with anti-proliferative activities; and

CSP-L-1 (old total lipid preparation, aged lipids) and CSP-L-2 (new total lipid preparation), total lipid fractions (extracted from the catfish skin preparation (CSP)).

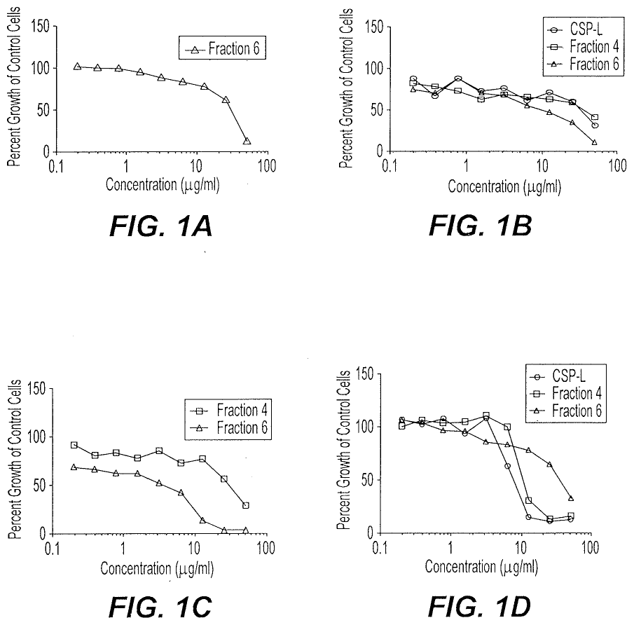

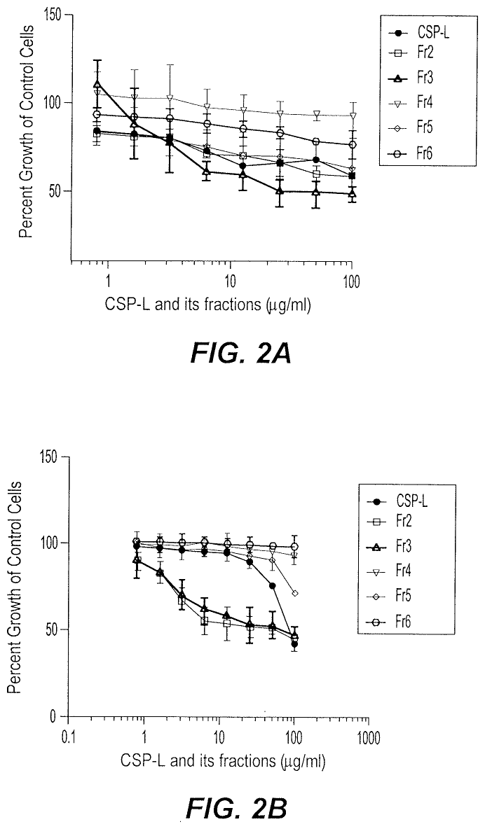

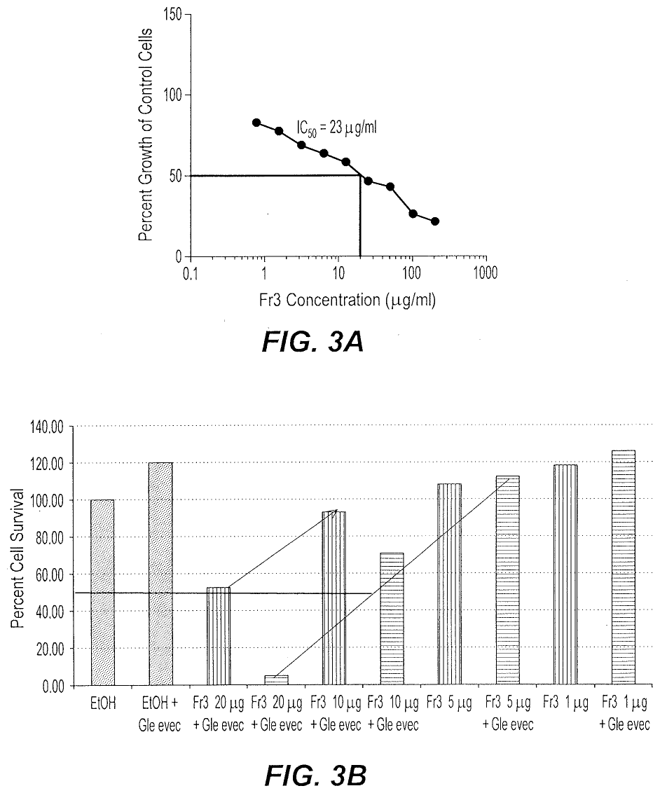

As described herein, each of the first, second, third, and fourth lipid fractions (i.e., lipid classes) can exhibit anti-inflammatory and/or anti-cancer activities. As shown in FIGS. 1A-ID, lipid fractions inhibit cell growth of human non-small cell lung cancer cells A549 (FIG. 1A), human pancreatic cancer cells Panc-1 (FIG. 1B), human prostate cancer cells LNCaP (FIG. 1C), human hepatocellular carcinoma cells Hep3B (FIG. 1D), melanoma MEWO cells (FIG. 3A) and leukemia cells (FIG. 3B). Accordingly, a therapeutic composition for treating cancer can include at least one of the first, second, third, and fourth lipid fractions (i.e., lipid classes).

As lipids are derived from an edible fish, the therapeutic composition is non-toxic unlike other available cancer-treating drugs, e.g., Gleevec, that are toxic. The composition can selectively target cancer cells in solid or non-solid tumors to cause cancer cell apoptosis and inhibit cancer cell proliferation without destroying other cells and without side effects. In an embodiment, at least one of the lipid fractions can cause apoptosis in solid tumors and/or non-solid tumors.

A method for treating cancer can include administering a therapeutically effective amount of the composition for treating cancer to a patient suffering from cancer. The cancer can be selected from at least one of lung cancer, liver cancer, pancreatic cancer, prostate cancer, skin cancer, breast cancer, and leukemia. The therapeutic composition can be administered to a patient in need thereof, by intraperitoneal (IP) injection or sub-cutaneous (SC) injection, orally or topically (in the case of skin cancer).

According to an embodiment, a method of obtaining the epidermal gelatinous secretion of catfish for preparing the lipid fractions can include collecting an epidermal gel secretion (EGS) from the skin of Arabian Gulf catfish (Arius bilineatus, Valenciennes) that is free from venom, vomit, feces, blood, or other contaminants from the fish, and extracting a lipid fraction from the freeze-dried epidermal gel secretions to provide the total lipid fraction which includes the neutral lipids, the glycolipids, and the phospholipids. The Arabian Gulf catfish naturally exudes a gelatinous secretion through its skin after the catfish is shocked, e.g., threatened or injured. For example, once a catfish is caught, it will struggle as it is towed to the surface with the fishing hook still in its mouth (as the catfish is a bottom dweller). As the fish reaches the surface, it struggles to defend itself and to escape the reduction in water pressure. This will cause the fish to secrete the EGS. Also, during its struggle, the catfish may secrete one or more contaminants, such as venom from its venom glands and dorsal and pectoral spines, feces from its anal pore, vomit from its mouth, and blood through its gills, if the fishing hook catches the gill rays. Shocking the fish can also be accomplished by thermal shock, physical abrasions, or neural stimulation, or simply by the action of towing it to the surface of the sea with the fishing hook in its mouth. The fish is washed thoroughly while it is still alive to remove contaminants. While the fish is still alive, the fish can be held through its gill cover to induce production of additional EGS. The EGS without any remaining contaminants on the skin can be collected by a gentle mechanical scraping or suction of the skin. The EGS is immediately frozen, e.g., in dry ice, then cooled to −80° C. (deep freeze) or kept frozen in liquid nitrogen, to limit microbial growth and prevent biochemical decomposition.

A pharmaceutical composition for treating cancer can include the therapeutic composition. To prepare the pharmaceutical composition, the therapeutic composition, as the active ingredient, is intimately admixed with a pharmaceutically acceptable carrier according to conventional pharmaceutical compounding techniques. Carriers are inert pharmaceutical excipients, including, but not limited to, binders, suspending agents, preservatives, and anti-oxidants. In preparing compositions in oral dosage form, any of the pharmaceutical carriers known in the art may be employed. For example, suitable carriers and additives include glycols, oils, flavoring agents, preservatives, starches, diluents, granulating agents, binders, encapsulation, nanotechnology carriers, and the like.

The present pharmaceutical compositions can be in unit dosage forms such as tablets, pills, capsules, powders, granules, ointments, creams, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The pharmaceutical compositions herein will contain, per dosage unit, e.g., tablet, capsule, powder, injection, teaspoonful, suppository and the like, an amount of the therapeutic composition necessary to deliver an effective dose.

The pharmaceutical composition can be a topical composition for treating human melanoma. The topical composition can include total lipids (lipid fraction 1) or at least a fraction eluted therefrom, or any compound isolated from one or more of the separated fractions. The topical composition can include a diluent, such as an aqueous cream. In an embodiment, the aqueous cream can include Unguentum Emulsificans Aquasum (UEA), emulsifying ointment (e.g., 300 g (or ratio of)); phenoxyethanol (e.g., 10 g (or ratio of)); and purified (distilled) water (e.g., 1000 g (or ratio of)). At least one of the extracted fractions, or any compound isolated therefrom can be mixed thoroughly with the diluent according to a desired ratio. In an embodiment, a topical composition for treating human melanoma can include the neutral lipid fraction and a diluent. The topical composition can include a concentration of 100 μg lipid fraction/g UEA.

The pharmaceutical composition can include an anti-cancer oral composition. In an embodiment, the oral composition can be administered in capsules. The oral composition can include the total lipid fraction, at least one fraction extracted therefrom, or any compound isolated from one or more of the extracted fractions. The oral composition can further include an edible oil (e.g., olive oil as a diluent). Exemplary anti-cancer oral formulations and the corresponding cancer type for which they can be used are provided below:

1. Human lung cancer cells type A549: 100 μg/g (Lipid Fraction 6: olive oil);

2. Human prostate cancer cells type Lncap cell: 50 μg/g (Lipid Fraction 6: olive oil) or 25 μg/g (Lipid Fraction 4: olive oil);

3. Human pancreatic cancer cells type Panc-1 and type-Capan-2: 50 μg/g (Lipid Fraction 6: olive oil), 50 μg/g (Lipid Fraction 4: olive oil), or 50 μg/g (Total Lipid, CSP-L: olive oil);

4. Human liver cancer cells Hep3B: 25 μg/g (Lipid Fraction 4: olive oil), 12.5 μg/g (Fraction 1, CSP-L: olive oil), or 60 μg/g (Lipid Fraction 6: olive oil); and

5. Human leukemia cells Type K562: 20 μg Fraction 3/μL ethanol+2 μM gleevec (a drug used to treat leukemia).

The following examples illustrate the present teachings.

### Example 1

### Total Lipid Extract, Fraction 1 (CSP-L)

Epidermal gel secretions (EGS) were collected from Arabian Gulf catfish (Arius bilineatus, Valenciennes) while the fish was still alive and immediately frozen, e.g., in dry ice, then cooled to −80° C. (deep freeze) or kept frozen in liquid nitrogen, to limit microbial growth and prevent biochemical decomposition. The collected EGS were freeze-dried. The resulting dry material was then kept under nitrogen in the dark in a freezer at −80° C. until use.

The total lipid fraction (Fraction 1) of the freeze-dried gel was obtained by extraction with chloroform: methanol: isopropanol (2:1:0.250, v/v) for 72 hours using a magnetic stirrer. The extracted lipids were obtained by filtration using a vacuum pump and a Buchner funnel. The residue (proteins) was re-extracted for at least four times using the same solvent mixture to collect the total lipid material (CSP-L, Fraction 1) present in the freeze-dried material. The residual proteins were saved for other applications.

The extracted lipid fractions were combined and concentrated to dryness on a rotary evaporator at 25° C. in the dark to provide the total lipid fraction. The residual material (total lipid fraction, Fraction 1) was re-dissolved in a minimum volume of chloroform: methanol (2:1 v/v) mixture for lipid fractionation on column chromatography.

The obtained total lipid fraction (CSP-L, Fraction 1) contained neutral lipids, glycolipids, and phospholipids. The total extracted lipids were dried of organic solvents under vacuum in the dark and stored under nitrogen at −80° C. in the dark until use. All the above steps should preferably be carried out in the dark to avoid any possible chemical changes to the lipids due to the combined effects of light and atmospheric oxygen. A cold room can be used for all these steps as well.

### Example 2

### Fraction 2 Lipids, the Neutral Lipids

The total lipid extract (Fraction 1) was applied to a 30×2 cm Woelm Pharma silica gel column (70-100 mesh, 100-200 μm particle size), packed in petroleum ether. The column was first eluted with 1 L petroleum ether. The elution was continued by the addition of an increasing amount of chloroform in petroleum ether in a linear gradient fashion as follows:

a) Petroleum ether, 1 liter

b) Petroleum ether: chloroform (75:25, v/v) 250 ml (to provide fraction 3 of the neutral lipids);

c) Petroleum ether: chloroform (50:50, v/v) 500 ml (to provide a fraction of the neutral lipids); and

d) Chloroform (100%) 1 liter (to provide fraction 4 of the neutral lipids).

The eluted solvents (obtained from steps a-d) were pooled and concentrated on a rotary evaporator to produce Fraction 2 Lipids. This lipid fraction was the neutral lipid fraction and was stored under nitrogen in the dark at −80° C.

### Example 3

### Fraction 6 Lipids, the Glycolipids

Elution of the column as described in Example 2 was continued with an increasing amount of acetone in chloroform in a linear gradient fashion as follows:

a) Chloroform: acetone (90:10 v/v) 1 liter);

b) Chloroform: acetone (75:25, v/v) 250 ml (to provide fraction 5);

c) Chloroform: acetone (50:50, v/v) 500 ml (to provide a fraction of the glycolipids); and

d) Acetone (100%) 1 liter (to provide a fraction of the glycolipids).

The eluted organic solvents (obtained from steps a-d) were pooled and concentrated on a rotary evaporator at 25° C. to produce Fraction 6 Lipids. This lipid fraction was the glycolipid fraction. It was stored under nitrogen in the dark at −80° C.

### Example 4

### Fraction 7 Lipids, the Phospholipids

Elution of the column as described in Example 3 was continued using increasing amounts of methanol in acetone in a linear gradient fashion as follows:

a) Acetone: methanol (90:10, v/v) 1 liter;

b) Acetone: methanol (75:25, v/v) 250 ml (to obtain a fraction of the phospholipids);

c) Acetone: methanol (50:50, v/v) 500 ml (to obtain a fraction of the phospholipids); and

d) Methanol (100%) 1 liter (to obtain a fraction of the phospholipids).

The organic solvents (obtained from steps a-d) were pooled and concentrated on a rotary evaporator to produce Fraction 7 Lipids. This lipid fraction was the phospholipid fraction. It was stored under nitrogen in the dark at −80° C.

### Example 5

### Anti-Cancer Topical Composition

A topical composition for treating human melanoma was prepared using the following aqueous cream as diluent and as a cream base:

Unguentum Emulsificans Aquasum (UEA) includes the following:

| | |
|---|---|
| a) Emulsifying ointment | 300 g (or ratio of); |
| b) Phenoxyethanol | 10 g (or ratio of); and |

c) Purified (distilled) water, freshly boiled and cooled to 1000 g (or ratio
of).

The components above were mixed thoroughly to provide the diluent and stored in the dark at 25° C.

For the composition for treating human melanoma, lipid fraction 3 of the neutral lipids was added to the diluent and mixed thoroughly with a concentration of 100 μg/g of the chosen fish lipid fraction or components (fish lipid: (UEA)). The resulting cream was applied thinly over the affected area after cleaning with absolute ethanol. FIG. 3A shows the effect of fraction 3 of the neutral lipids on human melanoma (MEWO) cells.

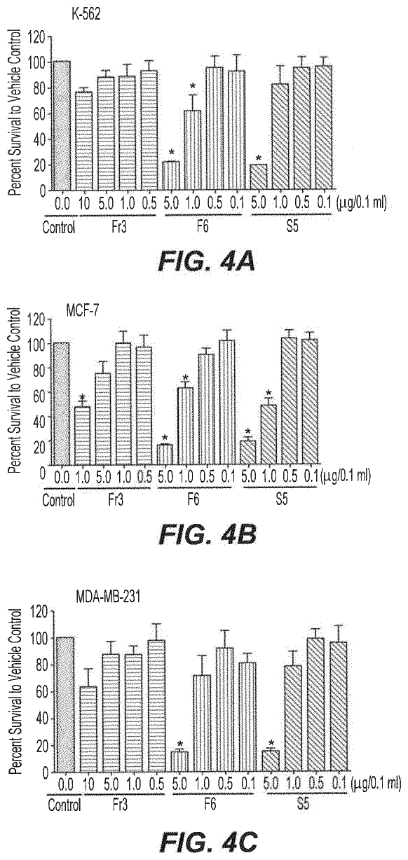

FIGS. 4A-4C show Fraction 3 of the neutral lipids, a furan (F6), and a steroid (S5) found in Fraction 3 of the neutral lipids and the two isolated components from Fraction 3 of the neutral lipids to be responsible for its anti-proliferative actions on 3 human cancer cell lines (leukemic K562, breast MCF-7 and breast MDA MB-231). The graphs show results of different concentrations of the compounds using the WST-1 spectrophotometric detection assay. WST is a colorimetric assay to assess cell proliferation under different treatment conditions. This assay is based on the cleavage of a tetrazolium salt (2-(4-Iodophenyl)-3-(4-nitrophenyl)-5-phenyl-2h-tetrazolium chloride) to form formazan (a colored compound) by mitochondrial dehydrogenases in viable cells. As a result, the assay measures the net metabolic activity of the cells. Human leukemia K562 cell line was purchased from ATCC (Manassas, Va., USA) and plated a day before the experiment in normal growth medium. The next day, the medium was replaced with serum free medium (Gibco #11835-030) to starve the cells for 4 h. Cells were deprived of serum for 4 h before treatment with the lipids to eliminate binding of the compounds to serum proteins. After starvation, K562 cells were then centrifuged. The medium was replaced with fresh IMDM media containing 1% (v/v) FBS and 100 units of PS. Cells (1×104 in 100 L) were seeded into each well with conditioned medium for overnight (20 h) in a 96-well plate to which the test compounds (−ve control ethanol, Ft-3, F-6 and S5) were added at the concentrations indicated. A volume of 10 uL WST-1, cell proliferation assay reagent (Roche Diagnostics, Indianapolis, Ind., USA), was added to each well and incubated for 4 h, and then the wells were read on a plate reader at 450 nm with a reference reading at 650 nm. Reference reading at 650 nm was used for subtracting background readouts.

FIG. 4B is a graph showing the effect of fraction 3 of the neutral lipids, furan (F6), and a steroid found in fraction 3 of the neutral lipids, (S5) and the two isolated components from fraction 3 of the neutral lipids on breast MCF-7 cells using the WST-1 spectrophotometric detection assay. WST was performed to assess cell proliferation under different treatment conditions. The assay measured the net metabolic activity of the cells. Human breast MCF-7 cell line was purchased from ATCC (Manassas, Va., USA). 1×104 cells/well/100 L) and were plated a day before experiment in normal growth medium. The next day, the medium was replaced with serum free medium (Gibco #11835-030) to starve the cells for 4 h. Cells were deprived of serum for 4 h before treatment with the lipids to eliminate binding of the compounds to serum proteins. After starvation, the medium was replaced with fresh IMDM media containing 1% (v/v) FBS and 100 units of PS. Cells (1×104 in 100 L) were seeded into each well with conditioned medium overnight (20 h) in a 96-well plate to which the test compounds (−ve control ethanol, Fr-3 of the neutral lipids, F6 and S5) were added at the concentrations indicated. A volume of 10 uL WST-1, cell proliferation assay reagent (Roche Diagnostics, Indianapolis, Ind., USA), was added to each well and incubated for 4 h. Then, the wells were read on a plate reader at 450 nm with a reference reading at 650 nm. Reference reading at 650 nm was used for subtracting background readouts.

FIG. 4C is a graph showing the effect of fraction 3 of the neutral lipids, furan (F6), and a steroid (S5) on human breast cancer cells MDA MB-231 using the WST-1 spectrophotometric detection assay. The assay measured the net metabolic activity of the cells. Human breast MDA MB-231 cell line was purchased from ATCC (Manassas, Va., USA). MCF-7 and 1_x 104 cells/well/100 uL) and were plated a day before experiment in normal growth medium. The next day, medium was replaced with serum free medium (Gibco #11835-030) to starve the cells for 4 h. Cells were deprived of serum for 4 h before treatment with the lipids to eliminate binding of the compounds to serum proteins. After starvation, Cells (1_x 104 in 100 L) were seeded into each well with conditioned medium overnight (20 h) in a 96-well plate to which the test compounds (−ve control ethanol, Ft-3, F-6 and S5) were added at the concentrations indicated. A volume of 10 uL WST-1, cell proliferation assay reagent (Roche Diagnostics, Indianapolis, Ind., USA), was added to each well and incubated for 4 h. Then, the wells were read on a plate reader at 450 nm with a reference reading at 650 nm. Reference reading at 650 nm was used for subtracting background readouts.

### Example 6

### Anti-Cancer Oral Composition

It should be understood that compositions including olive oil in the Examples described herein were not tested on cell lines. For tests conducted on cell lines, the lipid fraction or components from the lipid fractions were dissolved in solvents and added to media to form solutions that can allow the lipids to act on the cancer cell lines. Olive oil is not a suitable solvent for tests conducted on cell lines. For administration to humans, however, a carrier (a solvent) of the active lipids is required. Organic solvents or such media cannot be used for oral administration, as they are not suitable for human consumption and might cause harm to the patient. As such, olive oil can be used as the carrier (solvent). The active lipids can dissolve in olive oil and olive oil is safe for human consumption.

An anti-cancer oral composition was prepared by dissolving at least one of the lipid fractions 1-7, or a component separated therefrom in olive oil.

Various embodiments of the oral composition were prepared. A first embodiment of the anti-cancer oral composition was prepared by combining lipid fraction 6 and olive oil in a ratio of 100 μg/g. In another embodiment, the composition included lipid fraction 4 and olive oil in a ratio of 25 μg/g. These embodiments were based on tests done on cancer cell lines using these fractions (without olive oil). The active ingredients in Fr 4 and Fr 6 are components of the neutral lipids (F6 and S5) as detected and isolated from the neutral lipid fractions. They both act as potent anti-cancer and anti-inflammatory agents. Being anti-inflammatory, they eliminate inflammation, which is a major factor involved in cancer and leads to proliferation of cancer cells. As these potent active lipids are apoptotic on cancer cells, they will act on cancer cells to destroy them. The active lipids were tested for treating or inhibiting lung cancer type A549 cell line, human Panc-1 cells, human prostate cancer LNCaP cells and hepatocellular carcinoma cells Hep3B as shown in FIGS. 1A-1D.

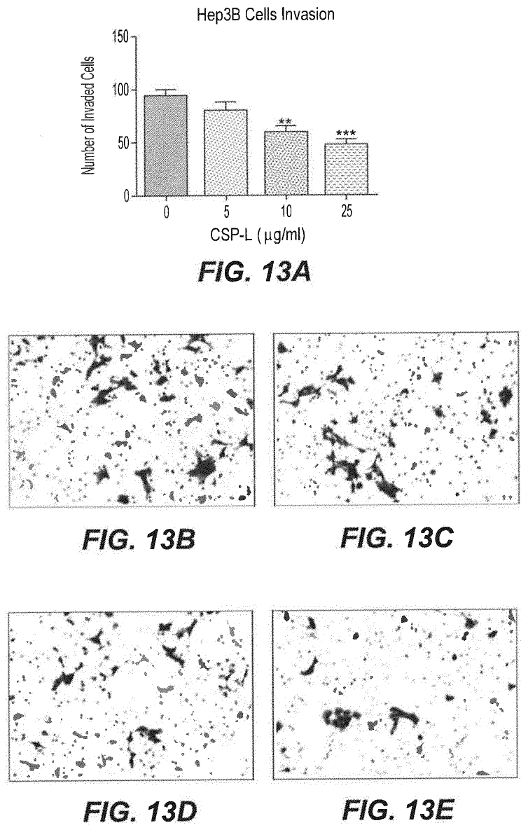

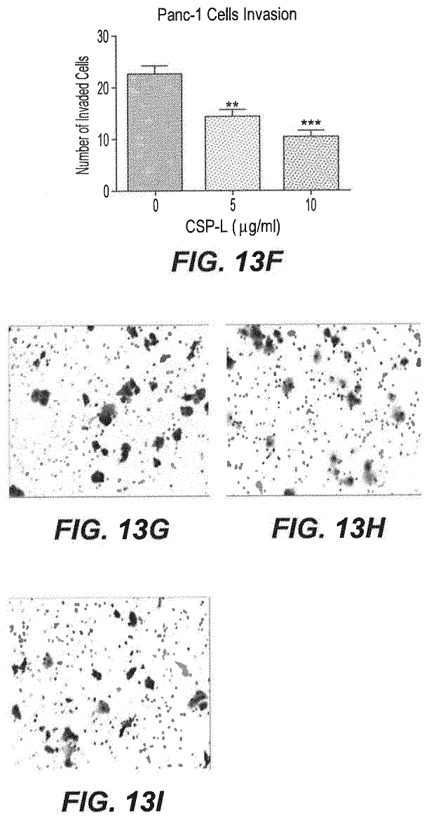

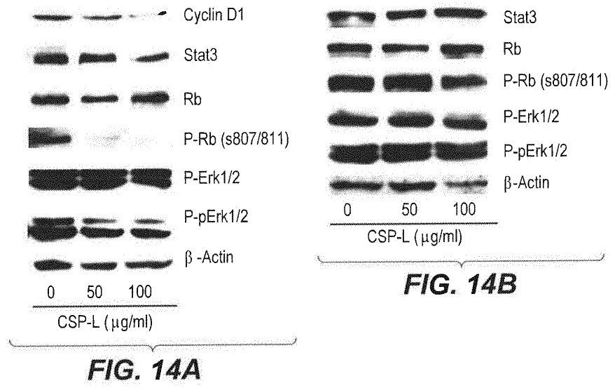

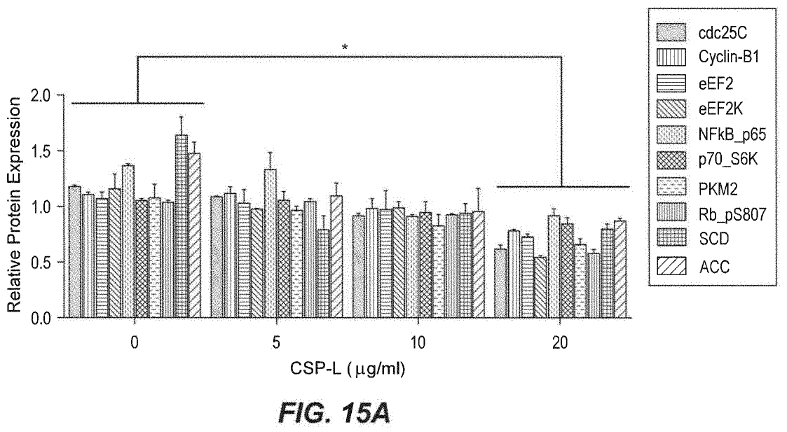

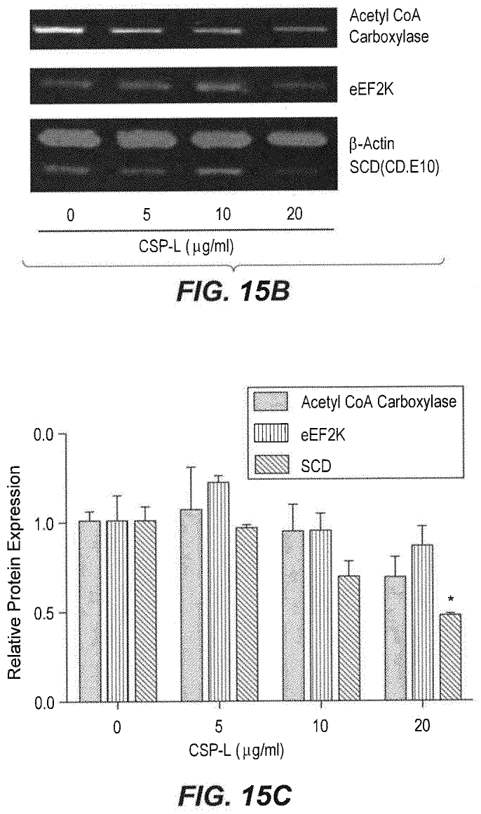

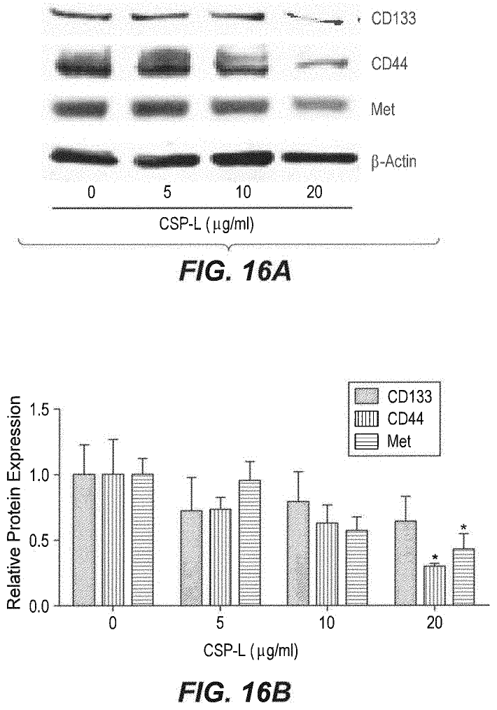

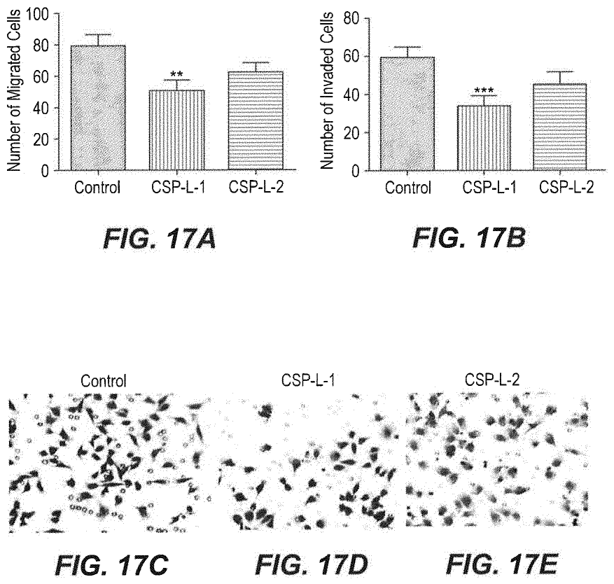

The effects of these active lipids on lung cancer type A549 cell line, Hep3B cells, Panc-1 cells, melanoma MEWO cells, breast MCF-7 cells, breast MDA MB-231 cells, leukemia K562 cells are provided in FIGS. 1A-D, and, 2A-B, 3A-B, 4A-C, 5A-C, 6A-C, 7A-D), 8A-C, 9A-B, 10A-B, 1A-G, 12A-E. FIGS. 13A-1 show the metastatic potential of Fr1 on Hep3B cells and Panc-1 cells. FIGS. 14A-B show protein quantitation results indicating that Fr1(CSP-L) modified cell cycle and cell signaling proteins in (FIG. 14A) Hep3B and (FIG. 14B) Panc-1 cells. FIGS. 15A-C show that increasing Fr1 concentration inhibits protein abundance associated with the cell's normal activities and cell signaling in Panc-1 cells, and affects acetyl CoA Caboxylase, eEF2K and SCD in Panc-1 cells. FIGS. 16 A-B show that CSP-L alters stem cell markers in Panc-1 cells. FIGS. 17A-E show the inhibitory effect of CSP-L and CSP-L-2 on cell migration and on cell invasion. Two major active components F6 and S5 (3,5-cholestadiene) found in CSP-L1 and CSP-L-2 inhibit Hep3B cell metastasis.

FIGS. 18A-18F show that F6 found in Fraction 1 (Fr1, CSP-L) regulated pancreatic cancer cell growth by induction of apoptosis. F6 potently inhibited proliferation of human pancreatic cancer, Panc-1, Capan2 and BxPc3 cells when these cells were treated with F6 (5-50 g/ml) for 72 hrs. FIGS. 19A-B show the inhibited invasion of Panc-1 cells by F6 in a concentration manner. Similar results were achieved with Hep3B (results are not shown here).

Another embodiment of the anti-cancer oral composition was prepared by combining lipid fraction 6 and olive oil in a ratio of 50 μg/g. In another embodiment, the composition included lipid Fraction 4 and olive oil in a ratio of 25 μg/g.

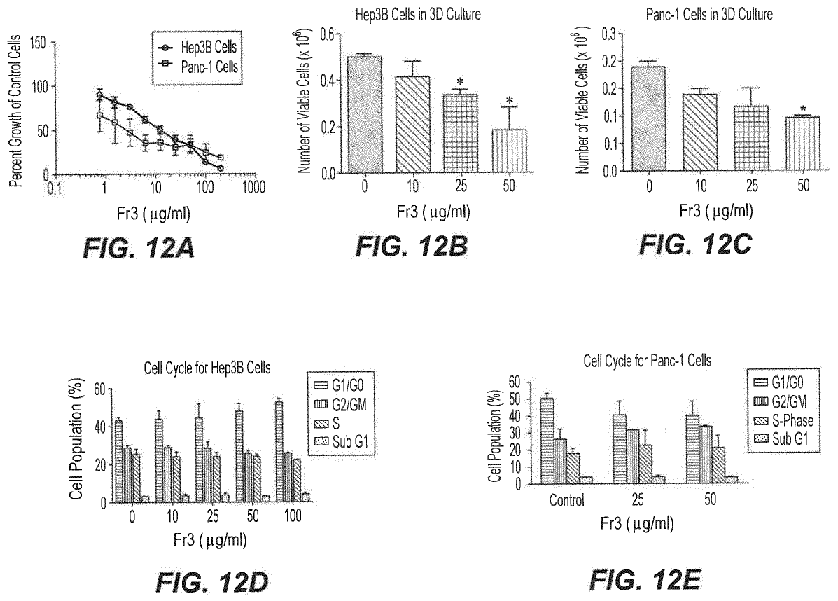

Another embodiment of the anti-cancer oral composition was prepared by combining lipid Fraction 6 and olive oil in a ratio of 50 μg/g. In another embodiment, the composition included the Total Lipid Fraction (Lipid Fraction 1) and olive oil in a ratio of 50 μg/g. These embodiments of the composition are suitable for treating or inhibiting pancreatic cancer. The effects of these active lipids on pancreatic cancer type Panc-1 cell line are provided in FIGS. 1B, 12A, 12C, and 12E.

Another embodiment of the anti-cancer oral composition was prepared by combining lipid Fraction 3 of the neutral lipids and olive oil in a ratio of 25 μg/g. In another embodiment, the composition included Fraction 1 and olive oil in a ratio of 12.5 μg/g. In another embodiment, the composition included Fraction 6 and olive oil in a ratio of 60 μg/g. These embodiments of the composition were tested for treating or inhibiting liver cancer type Hep3B cell line. The effects of these embodiments of the composition on liver cancer type Hep3B cell lines are provided in FIGS. 1D, 11B, 12A, 12B, 12D, and 14A.

Another embodiment of the anti-cancer oral composition was prepared by combining lipid Fraction 1 with ethanol to provide a first mixture in a ratio of 5 μg/mL, a second mixture in a ratio of 10 μg/mL, and a third mixture in a ratio of 25 μg/mL. The mixtures and a control (0.1% ethanol) were tested for inhibition of metastatic potential of cancer Hep3B cell line (FIGS. 13A-13E) and cancer Panc-1 cell line (FIGS. 13F-131). Less cell invasion was found in the presence of Fraction 1 as opposed to control in Hep3B cell line (FIGS. 13A-13E) and Panc-1 cell line (FIGS. 13F-131). Protein quantitation results showed that Fraction 1 modified cell cycle and cell signaling proteins in Hep3B (FIG. 14A) and Panc-1 cells (14B). Increased concentration of Fraction 1 inhibited protein abundance associated with normal cell activities, such as cell cycle, metabolism, and cell signaling in Panc-1 cells (FIG. 15A), and altered protein levels measured by protein quantification array (FIGS. 15B-15C). Further, Fraction 1 altered cancer stem cell markers in Panc-1 cells (FIGS. 16A-16B). Two major compounds, F6 and 3,5-cholestadiene, found in CSP-L-1 and CSP-L-2, inhibit cancer Hep3B cell metastasis (FIGS. 17A-17E).

Another embodiment of the anti-cancer oral composition was prepared by combining lipid Fraction 3 of the neutral lipid fraction and ethanol in a ratio of 20 μg/μL to provide a mixture. The mixture was prepared by dissolving Fraction 3 in pure ethanol then diluting it to the desired concentration with culture medium. Cells (1×10⁴) were starved overnight in serum-free medium, then the medium was replaced prior to incubation with IMDM/1% FBS/pen-strep. Doses of Fraction 3 of the neutral lipid fraction represent amounts added in ug/100 ul volume wells. The mixture was tested alone and in combination with 2 μm Gleevec for treating or inhibiting human leukemia type K562. The effect of lipid Fraction 3 of the neutral lipid fraction on the survival of human leukemic K562 cells and the response to ethanol in the absence and presence of Gleevec are shown in FIG. 3B as a function of an indicated dose. Gleevec at 2 uM potentiated the anti-proliferative effect of Fraction 3 of the neutral lipid fraction. The cell proliferation assay employed the WST-1 reagent colorimetric method that detects metabolically active cells.

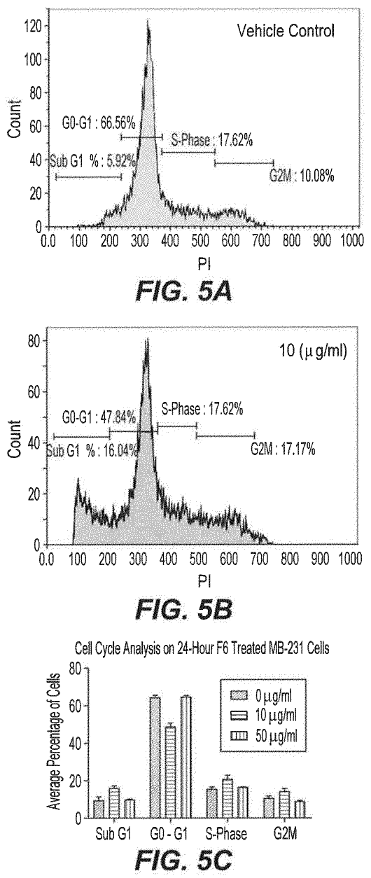

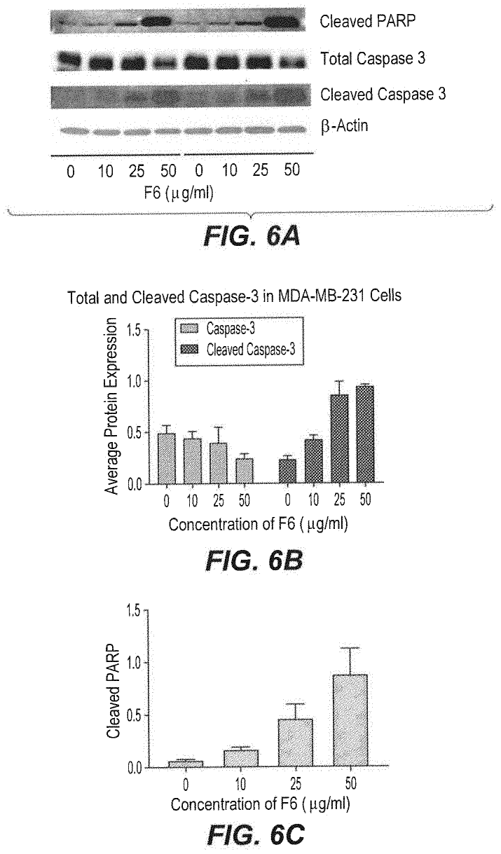

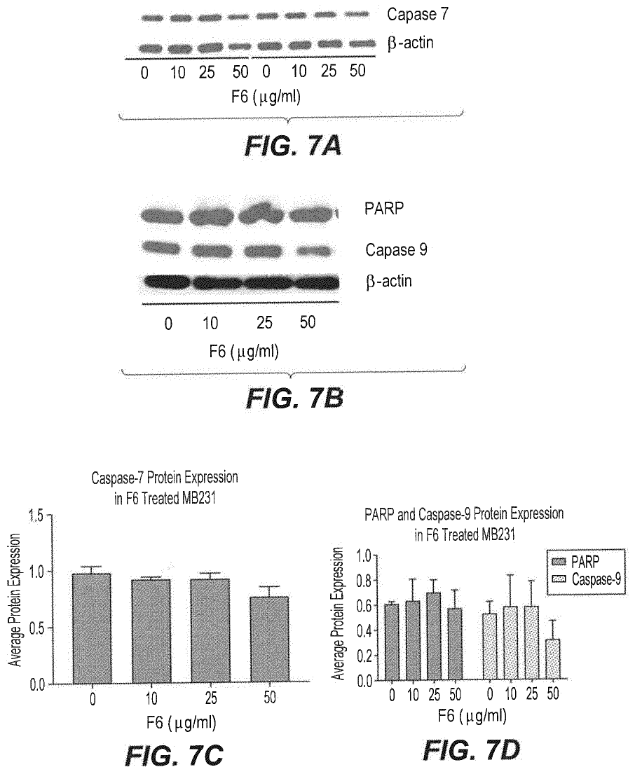

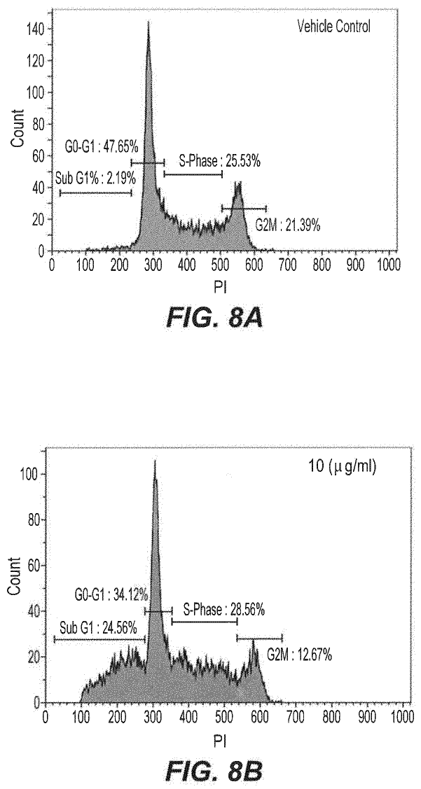

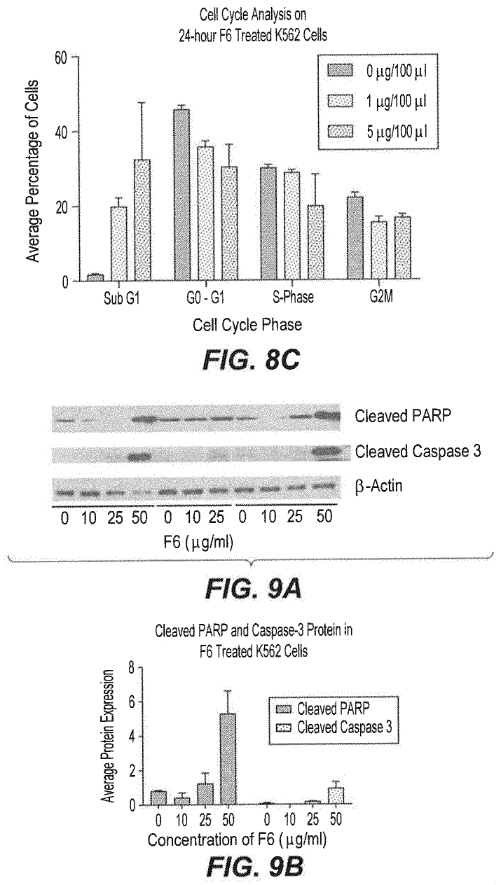

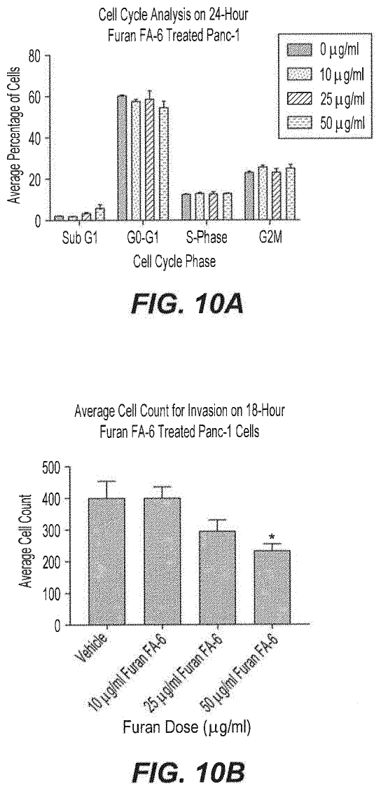

Furan fatty acid (F6), a lipid component found in the neutral lipid fractions, Fr-3 and Fr-4, was effective in inhibiting growth of cancer cells. The effect of furan F6 on human breast cancer MDA-MB-231 cells is shown in FIGS. 5A-5C. The expression of proapoptotic proteins, cleaved PARP and cleaved caspase 3 in F6 treated MDA-MB-231 cells is shown in FIGS. 6A-6C. The expression of caspase 7 and caspase 9 in F6 treated MDA-MD-231 cells is shown in FIGS. 7A-7D. FIGS. 8A-8C provide a cell cycle analysis of human leukemia K562 cells treated with furan fatty acid (F6). The cells were treated with F6 for 24 hours. FIGS. 9A-9B show the expression of proapoptotic proteins cleaved PARP and cleaved caspase 3 in F6 treated K562 cells. FIG. 10A-provides a cell cycle analysis of Panc-1 cells treated with F6. FIG. 10B shows the average cell count for invasion of Panc-1 cells treated with F6 (*p<0.05 treated versus control).

FIGS. 4A-4C show that two lipid components in Fraction 3 (Fr3 of the neutral lipid fraction), a furan (F6), and S5 (a steroid) can separately provide anti-proliferative effects on 3 human cancer cell lines: leukemic K562 (FIG. 4A), breast MCF-7 (FIG. 4B), and breast MDA MB-231 (FIG. 4C). F6 can regulate human pancreatic cancer cell growth by induction of apoptosis. FIG. 18A shows that F6 potently inhibited proliferation of human pancreatic cancer, Panc-1, Capan2, and BxPC3 cells when these cells were treated with F6 (5-50 μg/ml) for 72 hours. FIGS. 18A-18F show the effect of F6 on cell cycle and expression of apoptotic proteins on Panc-1 cells.

An aliquot of 0.5 ml Hep3B or Panc-1 cell suspension in serum-free medium with BSA (1 mg/ml) containing 6×10⁴ cells/ml was mixed with F6 and then applied to each invasion chamber. An aliquot of 0.5 ml medium with 10% serum was loaded into the bottom of the well. After incubation for 6 hours, cells that invaded to the bottom surface of the transwell were fixed with 70% ethanol, stained with Diff Quik solution (Sysmex), and counted in five selected fields. As shown in FIGS. 19A-19B, F6 inhibited invasion of Panc-1 cells in a concentration dependent manner as shown by light microscopy examination.

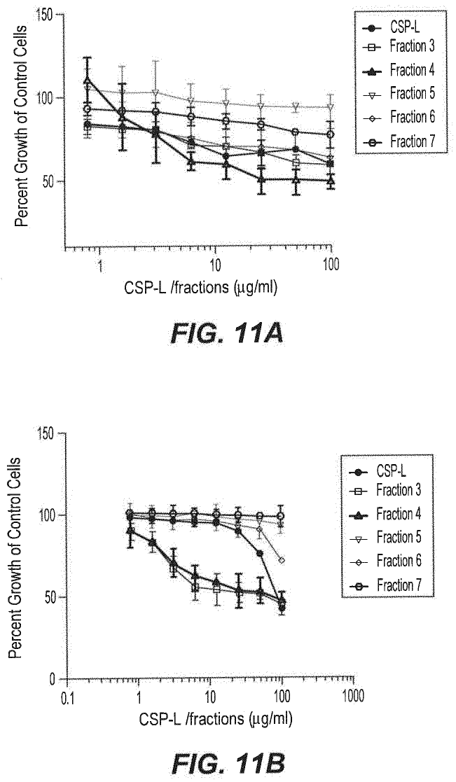

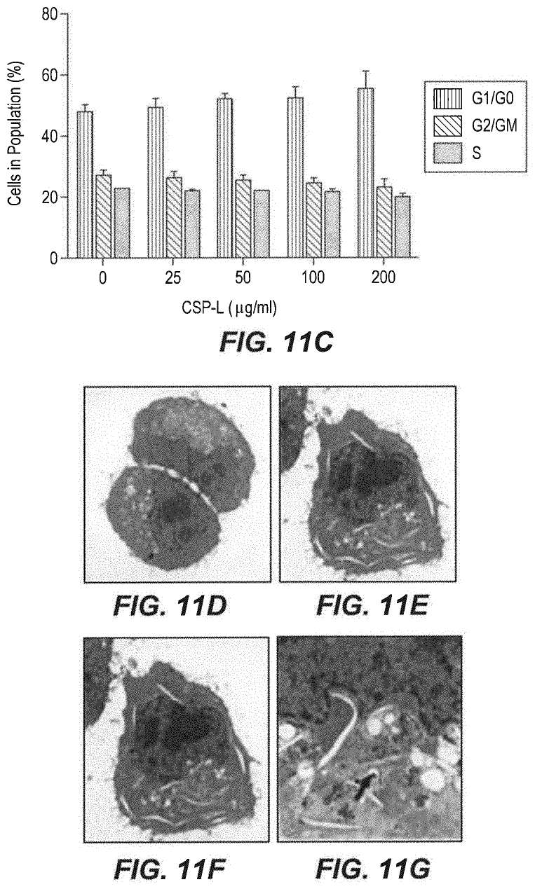

FIG. 11A shows Fraction 1 and Fractions derived therefrom (Fractions 3-7) reduce growth of human NSCLC A549 cells. FIG. 11B shows Fraction 1 and fractions derived therefrom (Fractions 3-7) reduce growth of Hep3B cells. FIG. 11C shows that Fraction 1 inhibited proliferation of Hep3B cells by arresting the cells at G1 growth phase. FIG. 11E (Fraction 1 treated cells; 5,000×) and 11G (Fraction 1 treated cells; 25,000×) are transmission electron microscope (TEM) images showing that cells treated with Fraction 1 presented with more signatures of cell death (more vacuoles and autophagosomes) than the control treated cells (FIGS. 11D (5,000×) and 11F (25,000×). It should be understood that each of the lipid fractions (the neutral lipid fraction, the glycolipid fraction, and the phospholipid fraction) include a number of lipid components. Further fractionation and purification using chromatography techniques, such as, High Pressure Liquid Chromatography (HPLC) and Preparative Liquid Chromatography, could be employed. These techniques can provide components such as F6 and S5 from the neutral lipid fraction. There are several other furans present in the F6, but in very small quantities. Because of their small concentrations, it is difficult to assign biological activities to these individual furan acids.

It is to be understood that the method of preparing a composition for treating cancer is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of preparing a composition for treating cancer, comprising:
collecting epidermal gel secretions of catfish;
freeze drying the collected epidermal gel secretions to obtain freeze-dried epidermal gel secretions; and
extracting a total lipid fraction from the freeze-dried epidermal gel secretions, the total lipid fraction including neutral lipids, glycolipids, and phospholipids,
wherein the composition for treating cancer comprises the total lipid fraction or a lipid fraction or lipid component fractionated from the total lipids.

2. The method of claim 1, wherein the extraction employs an extraction solvent mixture comprising chloroform, methanol, and isopropanol in a 2:1:0.25 ratio by volume.

3. The method of claim 1, further comprising filtration of the total lipid fraction and evaporating the solvent mixture of the filtered total lipid fraction to provide a final total lipid fraction.

4. The method of claim 3 further comprising chromatographic separation of three lipid classes from the final total lipid fraction, the three lipid classes being neutral lipids, glycolipids, and phospholipids.

5. The method of claim 4, wherein the chromatographic separation comprises:
dispensing a slurry of silica gel in petroleum ether in a chromatography column to provide a stationary phase for separating lipids;
loading the final total lipid fraction onto the stationary phase; and
separating three lipid classes from the final total lipid fraction consecutively using a different solvent system for each lipid class.

6. The method of claim 5, wherein separating the three lipid classes comprises eluting a neutral lipid fraction by dispensing a first solvent system into the chromatography column, the first solvent system comprising a non-polar solvent.

7. The method of claim 6, wherein an amount of the polar solvent in the first solvent system is gradually increased while eluting the neutral lipid fraction.

8. The method of claim 7 wherein eluting the neutral lipid fraction comprises:
a first eluting phase, wherein the first solvent system comprises 100% petroleum ether;
a second eluting phase after the first eluting phase, wherein chloroform is added to the first solvent system in an amount sufficient to achieve a petroleum ether: chloroform ratio by volume of 75:25 in the first solvent system;
a third eluting phase after the second eluting phase, wherein chloroform is added to the first solvent system in an amount sufficient to achieve a petroleum ether: chloroform ratio by volume of 50:50 in the first solvent system;
a fourth eluting phase after the third eluting phase, wherein chloroform is added to the first solvent system in an amount sufficient to achieve 100% chloroform in the first solvent system.

9. The method according to claim 8, wherein a solvent is eluted at each of the first, second, third, and fourth elution phases and the eluted solvents are pooled together and evaporated to provide the neutral lipid fraction.

10. The method of claim 6, wherein separating the three lipid classes comprises eluting a glycolipid fraction after eluting the neutral lipid fraction by dispensing a second solvent system into the chromatography column.

11. The method according to claim 10, wherein eluting the glycolipid fraction comprises:
a first eluting phase, wherein the second solvent system comprises a chloroform:acetone ratio by volume of 90:10;
a second eluting phase after the first eluting phase, wherein acetone is added to the second solvent system in an amount sufficient to achieve a chloroform:acetone ratio by volume of 75:25 in the second solvent system;
a third eluting phase after the second eluting phase, wherein acetone is added to the second solvent system in an amount sufficient to achieve a chloroform:acetone ratio by volume of 50:50 in the second solvent system;
a fourth eluting phase after the third eluting phase, wherein acetone is added to the second solvent system in an amount sufficient to achieve 100% acetone in the second solvent system.

12. The method according to claim 11, wherein a solvent is eluted at each of the first, second, third, and fourth elution phases and the eluted solvents are pooled together and evaporated to provide the glycolipid fraction.

13. The method according to claim 10, wherein separating the three lipid classes comprises eluting a phospholipid lipid fraction after eluting the glycolipid fraction by dispensing a third solvent system into the chromatography column, the third solvent system comprising methanol.

15. The method according to claim 10, wherein eluting the phospholipid fraction comprises:
a first eluting phase, wherein the third solvent system comprises 100% acetone;
a second eluting phase after the first eluting phase, wherein methanol is added to the third solvent system in an amount sufficient to achieve an acetone:methanol ratio by volume of 75:25 in the third solvent system;
a third eluting phase after the second eluting phase, wherein methanol is added to the third solvent system in an amount sufficient to achieve a acetone:methanol ratio by volume of 50:50 in the third solvent system;
a fourth eluting phase after the third eluting phase, wherein methanol is added to the third solvent system in an amount sufficient to achieve 100% methanol in the third solvent system.

16. The method according to claim 15, wherein a solvent is eluted at each of the first, second, third, and fourth elution phases and the eluted solvents are pooled together and evaporated to provide the phospholipid fraction.

17. A method for treating cancer, comprising administering a therapeutically effective amount of a composition for treating cancer to a patient in need thereof, the composition prepared by:
collecting epidermal gel secretions of catfish;
freeze drying the collected epidermal gel secretions to obtain freeze-dried epidermal gel secretions; and
extracting a total lipid fraction from the freeze-dried epidermal gel secretions, the total lipid fraction including neutral lipids, glycolipids, and phospholipids, the composition for treating cancer including the total lipid fraction or at least a fraction or a component eluted therefrom.

18. The method of treating cancer of claim 17, wherein preparing the composition further comprises eluting a neutral lipid fraction, a glycolipid fraction, and phospholipid fraction from the total lipid fraction, the composition for treating cancer including at least one of the neutral lipid fraction, the glycolipid lipid fraction and the phospholipid lipid fraction.
